(19) United States
(12) Patent Application Publication
(10) Pub. No.: US 2003/0059824 A1
(43) Pub. Date: Mar. 27, 2003

(54) Drug targets for alzheimer's disease and other diseases associated with decreased neuronal metabolism

(75) Inventor: Samuel T. Henderson, Broomfield, CO (US)

Correspondence Address: SWANSON & BRATSCHUN L.L.C., 1745 SHEA CENTER DRIVE, SUITE 330, HIGHLANDS RANCH, CO, 80129, US

(73) Assignee: ACCERA, INC.

(21) Appl. No.: 10/253,313

(22) Filed: Sep. 23, 2002

Related U.S. Application Data
(60) Provisional application No. 60/323,995, filed on Sep. 21, 2001.

(51) Int. Cl. C12Q 1/68; G01N 33/53; G01N 33/567

(52) U.S. Cl. 435/6; 435/7.2

(57) ABSTRACT

Target molecules for the development of assays and screening of compound libraries, which will be used to develop therapeutics for the prevention and treatment of Alzheimer's disease and other diseases associated with decreased neuronal metabolism are provided. Also provided are methods of treatment for the diseases.

### CROSS-REFERENCE TO RELATED APPLICATIONS

This application claims the benefit of U.S. Provisional Application No. 60/323,995, entitled “Drug Targets for Alzheimer's Disease and Other Diseases Associated with Decreased Neuronal Metabolism,” filed Sep. 21, 2001.

### FIELD OF THE INVENTION

This invention identifies target molecules for the development of assays and screening of compound libraries, which will be used to develop therapeutics for the prevention and treatment of Alzheimer's disease and other diseases associated with decreased neuronal metabolism.

### BACKGROUND OF THE INVENTION

Alzheimer's Disease

Alzheimer's disease (AD) is a progressive neurodegenerative disorder that primarily affects the elderly. In 1984, Blass and Zemcov proposed that AD resulted from a decreased metabolic rate in sub-populations of cholinergic neurons (Blass and Zemcov, Neurochem Pathol (1984) 2:103-14). The text of Blass and Zemcov, and the texts of all other patents and publications referred to herein, are incorporated by reference herein in their entirety. However, it has become clear that AD is not restricted to cholinergic systems, but involves many types of transmitter systems, and several discrete brain regions. The decreased metabolic rate appears to be related to decreases in glucose utilization. Brain imaging techniques have revealed decreased uptake of radiolabeled glucose in the brains of AD patients, and these defects can be detected well before clinical signs of dementia occur (Reiman, et al., N Engl J Med (1996) 334:752-8.). Measurements of cerebral glucose metabolism indicate that glucose metabolism is reduced 20-40% in AD resulting in critically low levels of ATP. The decreased metabolism is evident in the size and activity of cells. For example, certain populations of cells, such as somatostatin cells of the cortex, are smaller and have reduced Golgi apparatus (for review see (Swaab, et al., Prog Brain Res (1998) 117:343-77)).

Consistent with the decreased glucose metabolism, molecular components of insulin signaling and glucose utilization are impaired in AD (for review see (Swaab, et al., Prog Brain Res (1998) 117:343-77)). The brains of mammals are well supplied with receptors for insulin and IGF, especially in the areas of the cortex and hippocampus, which are important for learning and memory. In patients diagnosed with AD, increased densities of insulin receptors were observed in many brain regions, yet the level of tyrosine kinase activity was decreased, both relative to age-matched controls. The increased density of receptors represents up-regulation of receptor levels to compensate for decreased receptor activity. For example, activation of the insulin receptor is known to stimulate phosphatidylinositol-3 kinase (PI3K), and PI3K activity is reduced in AD. Furthermore, the density of the major glucose transporters in the brain, GLUT1 and GLUT3 were found to be 50% of age-matched controls. Importantly, while glucose utilization is impaired in AD, use of the ketone bodies beta-hydroxybutyrate and acetoacetate appears to be unaffected (Ogawa, et al., J Neurol Sci (1996) 139:78-82).

The cause of the decreased glucose metabolism remains uncertain, but may be related to processing of the amyloid precursor protein (APP). Mutations that alter the processing of APP have been implicated in early onset AD. Early onset cases occur before the age of 60 and in many cases have been associated with mutations in three genes: APP, presenilin 1 (PS1) and presenilin 2 (PS2). Mutations in these genes lead to aberrant processing of the APP protein (for review see (Selkoe, Nature (1999) 399:A23-31)). Where examined, these pathological mutations result in early defects in cerebral glucose metabolism. Individuals harboring a double mutation at APP670/671 (the “Swedish mutation”) exhibit pathological decreases in glucose metabolism in temporal lobes, often before clinical manifestations of dementia are evident. Mice carrying an APP V717F transgene exhibit regional defects in cerebral glucose metabolism. Also, mutations in the presenilin genes may directly increase susceptibility to glucose deprivation.

Yet, cases of early onset AD are rare, greater then 95% of cases of AD are late onset, and not associated with mutation in APP or PS1 or PS2. Late onset AD is associated with genetic risk factors and not well-defined genetic causes. One well-defined risk factor for late onset AD is allelic differences in apolipoprotein E gene. Presence of the epsilon4 (E4) variant of ApoE has been identified as a risk factor for late onset AD, yet the mechanism remains controversial. It may be related to interactions with Aβ, decreases in neuronal plasticity, or response to neuronal damage (for overview see (Strittmatter and Roses, Annu Rev Neurosci (1996) 19:53-77)).

The finding of decreased glucose metabolism in AD has generated numerous studies trying to link type II diabetes with AD. Results of these studies provide no clear association between the two conditions. In some studies diabetes is associated with increased risk of AD, while in others it decreased risk (for review see (Finch and Cohen, Exp Neurol (1997) 143:82-102)). These studies are no doubt complicated by numerous factors both in classification and biology. For example, without autopsy examination, it is difficult to distinguish between vascular dementia and AD. Also, because of the unique architecture and energy requirements of the brain, insulin action and glucose utilization differs greatly between the brain and other tissues. Genetic and environmental factors that give rise to insulin resistance in muscle may differ from those in the brain. Also, altered glucose and ketone body levels in diabetics may complicate the association. Peripheral insulin resistance may increase the risk of AD by a general disturbance in glucose homeostasis. Yet, under different conditions, insulin resistance may confer protection against AD by increasing ketone body levels in the blood. Interestingly, some studies have shown that type II diabetics not treated with insulin have a lower risk of AD, while those treated with insulin have a higher risk. Perhaps this is because insulin treatment suppresses ketone body production and no alternative to glucose is available to the brain.

Attempts to compensate for reduced cerebral metabolic rates in AD has met with some success. Treatment of AD patients with high doses of glucose and insulin will raise cognitive scores. However, this effect is slight, and high doses of insulin can have adverse consequences.

Ketone Bodies

Cerebral neurons can also utilize ketone bodies as an energy substrate. Ketone bodies serve a critical role in the development and health of cerebral neurons. Neonatal mammals are dependent on maternally derived milk for development. The major carbon source of milk is fat (only about 12% of the caloric content of milk is carbohydrate). The fatty acids in milk are partially oxidized to form ketone bodies, which fuel much of neonatal development, and in particular the brain. Numerous studies have shown that the preferred substrates for the developing mammalian neonatal brain are ketone bodies (for review see (Edmond, Can J Physiol Pharmacol (1992) 70:S118-29)). Ketone bodies also function in adult mammals. There is a large body of evidence demonstrating that ketone bodies are used in a concentration dependent manner by the adult human brain, even in the elderly. When systemic glucose levels are low, the liver produces large amounts of ketone bodies to fuel the body, and especially cerebral neurons. Cerebral neurons have a high metabolic rate and cannot efficiently oxidize fatty acids, and therefore rely on a continuous supply of glucose, lactate, or ketone bodies from the blood for proper function. In a normal Western diet, cerebral neurons are fueled almost exclusively by glucose. This leaves cerebral neurons susceptible to glucose shortages. If blood glucose levels drop rapidly, ketone bodies cannot be mobilized fast enough and damage occurs. Yet, if glucose levels are lowered slowly, such as during a fast, the liver mobilizes ketone bodies, and cerebral damage is averted. Interestingly, since glia can use a wider range of energy substrates, they are less susceptible to glucose shortages. This is consistent with the observation that in AD, neurons die, but glia do not.

In AD, glucose metabolism is reduced in the brain, but normal in peripheral tissues, hence the liver fails to mobilize ketone bodies. Without an alternative to glucose, cerebral neurons starve. Therefore it is the novel insight of this invention that induction of hyperketonemia may prove beneficial in AD, and other diseases associated with decreased glucose utilization.

Ketone bodies are produced from the partial oxidation of fatty acids by two major cell types: hepatocytes (liver cells) and astrocytes (neuronal support cells). The production of ketone bodies is regulated by several mechanisms in both hepatocytes and astrocytes.

Regulation of Fatty Acid Oxidation in the Liver

An overview of the regulation of the oxidation of Free Fatty Acids (FFA) in the liver is shown in FIG. 1 (for review see (Murray, et al., in Harper's Biochemistry (1999) 927)). FFA in hepatocytes are either esterified and assembled as triglycerides for distribution as VLDL particles, or they are oxidized in the mitochondria. For oxidation, FFA are first converted to Acyl-CoA molecules. These Acyl-CoA molecules cannot penetrate the mitochondria, therefore they are combined with carnitine to allow transport into the mitochondria by Carnitine Palmitoyl-Transferase I (CPTI). In the mitochondria, carnitine is removed and Acyl-CoA molecules undergo beta-oxidation. If large amount of FFA are being oxidized, more Acetyl-CoA is produced than can be used by the mitochondria, and the excess Acetyl-CoA is used to synthesize ketone bodies. Since the liver cannot use ketone bodies they are released into to the bloodstream to be used by extrahepatic tissues. The oxidation of fatty acids in the liver is mainly controlled by regulating entry of Acyl-CoA into the mitochondria. In a well fed state, excess Acetyl-CoA derived from carbohydrate sources are converted to Malonyl-CoA by the enzyme Acetyl-CoA Carboxylase (ACC) as a first step in lipogenesis. Malonyl-CoA is a potent inhibitor of Carnitine Palmitoyl-Transferase I and thereby blocks the entry of fats into the mitochondria. Several factors are known to influence the activity of ACC. Insulin is known to increase the activity of ACC, thereby promoting fat storage and inhibiting fat oxidation. Glucagon is known to inhibit ACC and promote fat oxidation.

Regulation of Fatty Acid Oxidation in Astrocytes

Astrocytes are neuronal support cells that insure health of cerebral neurons. An overview of the regulation of the oxidation of FFA in astrocytes is shown in FIG. 2 (for review see (Guzman and Blazquez, Trends Endocrinol Metab (2001) 12:169-73.)). It is believed that FFA entering astrocytes are either oxidized or used in the synthesis of ceramides. The control of the oxidation of FFA in astrocytes in similar to that seen in the liver (see above, and FIG. 1). Excess Acetyl-CoA is converted to Malonyl-CoA by Acetyl-CoA carboxylase (ACC), and Malonyl-CoA inhibits the Carnitine Palmitoyl-Transferase found in astrocytes. Additional regulation of fatty acids oxidation has been identified in astrocytes. Endocannabinoids, endogenous ligands for the cannabinoid receptors, have been shown to increase the production of ketone bodies by astrocytes (Guzman and Blazquez, Trends Endocrinol Metab (2001) 12:169-73.). Also AMP activated protein kinase (AMPK) is known to phosphorylate and thereby inactivate ACC. Decreased ACC activity reduces the amount of Malonyl-CoA which results in increased activity of Carnitine Palmitoyl-Transferase and increased fatty acid oxidation.

Uptake of Ketone Bodies

Once released into the bloodstream ketone bodies are transported into cells by monocarboxylate transporters (MCTs). MCTs are a family of proteins that transport a variety of monocarboxylic acids including, lactate, pyruvate, branched-chain oxo acids and ketone bodies. These transporters catalyze the facilitated diffusion of monocarboxylic acids across membranes with a proton. They require no energy input other then the concentration gradients of the protons and monocarboxylic acids. Therefore, the transport of ketone bodies depends on the concentration of ketone bodies, the pH gradient, and the number and activity of MCT proteins on cell surface. The blood brain barrier is relatively impermeable to monocarboxylic acids and therefore the rate of entry into the brain is dependent on the presence and activity of these transporters. MCTs are expressed in the brain and can be found in astrocyte footpads surrounding brain capillaries (for review see (Halestrap and Price, Biochem J (1999) 343 Pt 2:281-99).

The levels of MCTs in the brain change during development and in response to diet. MCT levels are high in neonatal mammals and decrease in adults. Suckling mammals are dependent on fat rich milk for much of development and require high levels of MCTs to transport ketone bodies into the brain. During these periods glucose is largely reserved for the pentose pathway for the production of nucleic acids and lipids. As the animal ages the brain switches to glucose for fuel and the levels of MCTs decrease. In an adult mammal, the level of MCTs in the brain is low, especially in the fed state when glucose in present in the plasma. However, the adult brain will use ketone bodies for fuel during periods of starvation or low carbohydrate intake, and under these conditions MCTs levels rise. For example, in rats fed a high-fat low-carbohydrate diet, the levels of MCT1 in the brain increases eight fold (Leino, et al., Neurochem Int (2001) 38:519-27). Therefore it is possible to up-regulate the number of MCTs in the adult mammalian brain, however this normally does not occur in the presence of abundant glucose.

### SUMMARY OF THE INVENTION

It is the novel insight of this invention that therapeutic compounds can be developed that increase the availability of ketone bodies to neurons, and that this increase in ketone body availability will be beneficial in Alzheimer's disease and other diseases associated with decreased cerebral glucose utilization.

The present invention provides a method of treating or preventing dementia of Alzheimer's type, or other loss of cognitive function caused by reduced neuronal or astrocyte cell metabolism, such as that caused by Parkinson's disease or Huntington's disease by increasing the availability of ketone bodies to neurons or astrocytes.

The present invention also provides a method of treating or preventing dementia of Alzheimer's type, or other loss of cognitive function caused by reduced neuronal or astrocyte cell metabolism, comprising increasing the output of ketone bodies by hepatocytes. This increase in the output of ketone bodies can be accomplished by modulating the activity of various targets in the metabolic pathway such as acetyl-CoA carboxylase, including inhibiting the action of insulin on acetyl-CoA carboxylase and modulating the intracellular level of glucagon. Increasing the output of ketone bodies can also comprise modulating the binding of malonyl-CoA to carnitine palmitoyl-transferase I, and increasing the availability of carnitine in hepatocytes.

The present invention also provides a method of treating or preventing dementia of Alzheimer's type, or other loss of cognitive function caused by reduced neuronal metabolism, comprising increasing the availability of ketone bodies to astrocytes. Increasing the availability of ketone bodies can be accomplished by modulating the activity of various targets in the metabolic pathway such as apoC2, including inhibiting E4 binding to VLDL. Another target is lipoprotein lipase, and modulation of its activity includes increasing C2 binding to VLDL. Another target is the cannabinoid receptor. Still other methods of increasing the availability of ketone bodies include modulating the binding of malonyl-CoA to carnitine palmitoyl-transferase I and increasing the availability of carnitine in astrocytes, modulating the activity of acetyl-CoA carboxylase, including modulating the activity of adenosine monophosphate kinase.

The present invention also provides a method of identifying an agent that increases the output of ketone bodies by hepatocytes comprising contacting a hepatic cell or hepatic-derived cell with at least one candidate agent, and detecting the output of ketone bodies by the cell, whereby the agent is identified. The detection can be performed at many levels, including genomic, transcriptional, protein or metabolic.

The method is used with candidate agents suspected of modulating the function of various metabolic targets. In one embodiment, the candidate agents is suspected of modulating the function of acetyl-CoA carboxylase, inlcuding inhibiting the action of insulin on acetyl-CoA carboxylase, and modulating the intracellular level of glucagon. In another embodiment, the candidate agent is suspected of modulating the binding of malonyl-CoA to carnitine palmitoyl-transferase I, or modulating the availability of carnitine.

The present invention also provides a method of identifying an agent that increases the output of ketone bodies by astrocytes comprising contacting an astrocyte or astrocyte-derived cell with at least one candidate agent, and detecting the output of ketone bodies or activity of the target of the cell.

The method is used with candidate agents suspected of modulating the function of various metabolic targets. In one embodiment, the candidate agent is suspected of modulating the function of apoC2, modulating the function of cannabinoid receptors, or modulating the function of lipoprotein lipase, modulating the binding of malonyl-CoA to carnitine palmitoyl-transferase I, modulating the availability of carnitine, modulating the activity of acetyl-CoA carboxylase, or modulating the activity of adenosine monophosphate kinase.

The present invention also provides a method of identifying an agent that increases the uptake of ketone bodies in the brain. In one embodiment, the candidate agent is suspected of modulating the function of monocarboxylate transporters (MCT).

The present invention further provides a method of identifying an agent that increases the uptake of ketone bodies by a component selected from the group consisting of an astrocyte, and astrocyte-derived cell, non-neonatal brain, and non-neonatal brain tissue, comprising contacting said component with at least one candidate agent, and detecting the uptake of ketone bodies by said component, whereby the agent is identified, including a method wherein the candidate agent is suspected of modulating the levels or activity of the monocarboxylate transporter family of proteins.

### BRIEF DESCRIPTION OF THE FIGURES

FIG. 1 shows regulation of fatty acid oxidation in the liver. Positve regulation is shown as + sign. Negative regulation is shown as − sign. FFA=Free Fatty Acids, VLDL=Very Low Density Lipoprotein.

FIG. 2 shows regulation of fatty acid oxidation in astrocytes. Positve regulation is shown as + sign. Negative regulation is shown as − sign. FFA=Free Fatty Acids, AMPK=AMP activated kinase.

FIGS. 3A and 3B show a model for the role of ApoE4 in AD. FIG. 3A shows VLDL particles are secreted by the liver to transport triglycerides. VLDL particles are bound by ApoE (E), and ApoC2 (C2). ApoC2 acts with lipoprotein lipase (LPL) on astrocytes to release free fatty acids (FFA). FFA enter astrocytes where they are oxidized to from ketone bodies which are shuttled to neurons as an energy source. FIG. 3B shows in ApoE4 carriers, E4 has higher affinity for VLDL particles and excludes C2 binding. Less C2 on VLDL particles reduces the activity of LPL on astrocytes, resulting in less FFA absorbed and less ketone body production by astrocytes.

### DETAILED DESCRIPTION OF THE INVENTION

This invention describes methods to increase ketone body availability to cerebral neurons. Increases in the availability of ketone bodies to neurons can be achieved by increasing the concentration of ketone bodies in the blood (hyperketonemia) or by increasing the production of ketone bodies by astrocytes. The targets listed below can be used in assays to identify compounds that will be useful in treating Alzheimer's disease and other diseases associated with decreased neuronal glucose utilization.

Without being bound to one particular theory of operation, it is believed that reduced neuronal metabolism is a component not only of Alzheimer's disease but of numerous other neurological disorders that result in a decrease in cognitive function. While Alzheimer's disease of the familial or the sporadic type is the major dementia found in the aging population, other types of dementia are also found. These include but are not limited to the fronto-temporal degeneration associated with Pick's disease, vascular dementia, senile dementia of Lewy body type, dementia of Parkinsonism with frontal atrophy, progressive supranuclear palsy and corticobasal degeneration and Downs syndrome associated Alzheimers', Multiple System Atrophy, Progressive Supranuclear Palsy, dementia as a result of neurosyphilis, dementia as a result of AIDS, dementia as a result of tumors, dementia as a result of brain injury, Huntington's disease, epilepsy, refractive epilepsy, Gilles de la Tourette's syndrome, autonomic function disorders such as hypertension and sleep disorders, and neuropsychiatric disorders that include, but are not limited to schizophrenia, schizoaffective disorder, attention deficit disorder, attention deficit hyperactivity disorder, dysthymic disorder, major depressive disorder, mania, obsessive-compulsive disorder, psychoactive substance use disorders, anxiety, panic disorder, as well as bipolar affective disorder, e.g., severe bipolar affective (mood) disorder (BP-I), bipolar affective (mood) disorder with hypomania and major depression (BP-II). Plaque formation is also seen in the spongiform encephalopathies such as CJD, scrapie and BSE. The present invention is directed to treatment of such neurodegenerative diseases.

The development of assays for the screening of compounds which modulate the function of the various targets is also an object of this invention. By “modulating the function” or “modulating the activity” it is meant altering when compared to not adding an agent. Modulation may occur on any level that affects function. A polynucleotide or polypeptide function may be direct or indirect, and measured directly or indirectly. Modulation may be an increase (stimulation) or a decrease (inhibition) in the function of the target.

1. Increasing Ketone Body Production by the Liver

The liver is the main site of ketone body production in humans. As described in the background section, long chain triglyerides (which make up the large majority of dietary triglycerides), are normally not oxidized to form ketone bodies. The regulation of the oxidation of fatty acids in the liver is shown in FIG. 1. It is the novel insight of this invention that alterations in this pathway will result in hyperketonemia, and that this state will be beneficial in treating and preventing Alzheimer's Disease and other diseases that exhibit decreased glucose metabolism.

a. Inhibition or Reduction in the Activity of Acetyl-CoA Carboxylase (ACC)

ACC converts cytoplasmic acetyl-CoA to Malonyl-CoA as an intermediate in the synthesis of lipids (lipogenesis). Malonyl-CoA is a potent inhibitor of Carnitine Palmitoyl-Transferase I (CPTI). Inhibition or reduction in the activity of ACC will decrease the cellular concentration of Malonyl-CoA, thereby increasing the activity of CPTI and the oxidation of fatty acids. Increasing the oxidation of fatty acids will lead to increased production of ketone bodies, and hyperketonemia.

b. Blocking Binding of Malonyl-CoA to CPTI

Malonyl-CoA binds to and inhibits the activity of CPTI. Blocking the binding of Malonyl-CoA to CPTI will increase the activity of CPTI, increase fatty acid oxidation and increase the production of ketone bodies, and lead to hyperketonemia.

c. Increase Intracellular Concentration of Carnitine

Long chain acyl-CoA cannot penetrate the outer mitochondrial membrane. Carnitine is attached to the Acyl-CoA chains to allow transport into mitochondria by CPTI and oxidation. Increasing the availability of carnitine will increase the activity of CPTI and increase oxidation of fatty acids, increase the production of ketone bodies, and lead to hyperketonemia.

d. Decrease Activity of Insulin

Insulin increases the activity of ACC thereby increasing the cytoplasmic concentration of Malonyl-CoA, which inhibits CPTI. Inhibiting the action of insulin increases ketone body production, which will be beneficial in treating and preventing Alzheimer's Disease and other diseases that exhibit decreased glucose metabolism.

e. Increase Activity or Levels of Glucagon

Glucagon inhibits the activity of ACC. Increasing the activity or intracellular concentration of glucagon will decrease the activity of ACC and decrease the production of Malonyl-CoA, resulting in increased ketone body production.

2. Increasing Ketone Body Production by Astrocytes

a. Increase the Activity of Apolipoprotein C2 and Lipoprotein Lipase

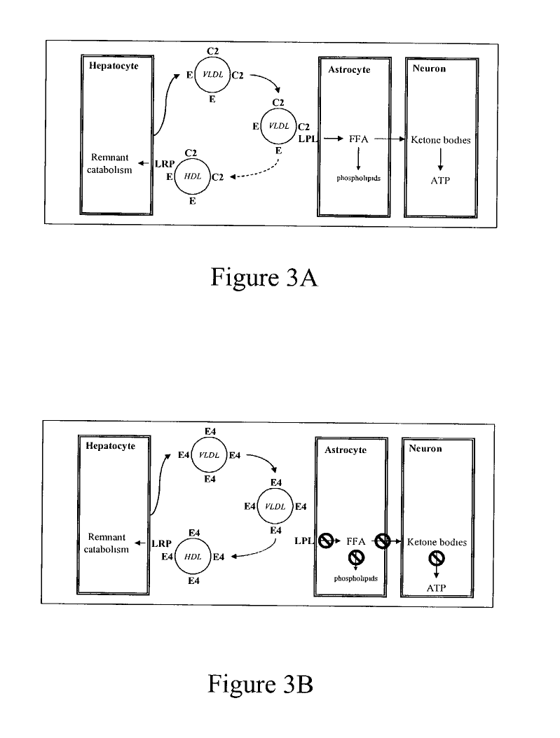

One of the major risk factors for late onset AD is possession of the epsilon 4 variant of the apolipoproteinE gene. The major function of ApoE is lipid transport. There are three major isoforms of apoE: E2, E3 and E4. Variations in the protein sequence of each form confer different functional characteristics. Recent studies suggest that the major difference between E4 and the other common isoforms is in the affinity for different lipoprotein complexes. E4 preferentially binds triglyceride rich particles (VLDL), while E2 and E3 preferentially bind HDL particles. The increased binding of E4 to VLDL particles blocks binding of apolipoprotein C2 (apoC2) resulting in less C2 bound to VLDL particles. C2 is a cofactor for lipoprotein lipase (LPL) that functions to cleave fatty acid chains from triglycerides in VLDL particles (see FIG. 3A). Since E4 blocks C2 binding, E4 decreases the rate of conversion of VLDL to higher density particles (for overview see (Mahley, et al., J Lipid Res (1999) 40:1933-49.)). Therefore, this decreased fatty acid usage may lead to the increased circulating VLDL and LDL levels seen in E4 carriers. It is the novel insight of this invention that this model can explain E4's role in AD (see FIG. 3B). Recent experiments have shown that astrocytes are capable of incompletely oxidizing fatty acids to yield ketone bodies, which are shuttled to cerebral neurons as an energy substrate (for review see (Guzman and Blazquez, Trends Endocrinol Metab (2001) 12:169-73.)). In E4 carriers, less C2 is bound to the VLDL particle and astrocytes can less efficiently cleave fatty acids from triglycerides resulting in fewer ketone bodies produced (3B). Ketone bodies provide an alternative energy source to cerebral neurons when glucose metabolism is compromised. Therefore, the decreased ability of astrocytes in E4 carriers to produce ketone bodies may accelerate the progression of AD. It is the novel insight of this invention that by increasing the levels or activity of apoC2 or lipoprotein lipase, astrocytes will produce more ketone bodies which can be shuttled to cerebral neurons for use as an energy substrate.

b. Activation of Cannabinoid Receptors

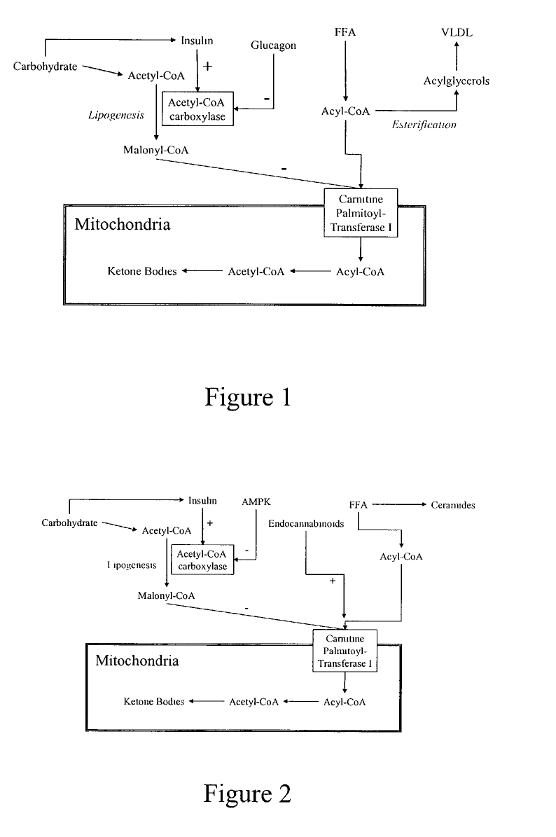

Endocannabinoids increase the production of ketone bodies by astrocytes (see FIG. 2). Therefore activation of cannabinoid receptors will increase the output of ketone bodies by astrocytes and provide an alternative energy substrate to cerebral neurons with compromised glucose metabolism, such as occurs in Alzheimer's Disease.

c. Increase Intracellular Concentrations of Carnitine

Long chain acyl-CoA cannot penetrate the outer mitochondrial membrane. Carnitine is attached to the acyl-CoA chains to allow transport into mitochondria by CPTI and oxidation. Increasing the availability of carnitine will increase the activity of CPTI and increase oxidation of fatty acids, increase the production of ketone bodies which can be shuttled to cerebral neurons.

d. Activate Adenosine Mono-Phosphate Kinase (AMPK)

Activation of AMPK inhibits ACC and decreases intracellular Malonyl-CoA concentration and thereby increases the oxidation of fatty acids in astrocytes, resulting in increased ketone body production.

3. Increasing Uptake of Ketone Bodies by the Brain

a. Monocarboxylate Transporters (MCT)

Ketone bodies are transported by facilitated diffusion into the brain using MCT. The consumption of a typical Western diet, rich in carbohydrates, results in low levels of MCT in the adult human brain. These low levels of MCTs limit the amount of ketone bodies that can be transferred to the brain. It is the novel insight of the inventor that agents that increase the activity or levels of the MCT protein will prove beneficial.

Conclusions Ramifications and Scope

Accordingly, the reader see that the targets described in this invention can be used to develop treatments and preventative measures for Alzheimer's disease, and other diseases associated with decreased neuronal metabolism. The invention describes methods to increase the availability of ketone bodies to neurons. In particular, it describes increasing the output of ketone bodies by hepatocytes (liver cells) and astrocytes (neuronal support cells). While Alzheimer's disease in the focus of the Background discussion it should not be considered a limitation of the invention. Other neurological disorders such as Parkinson's disease and Huntington's disease, which also exhibit decreased neuronal glucose metabolism, will benefit for the alterations in the pathways described.

A better understanding of the present invention may be obtained in light of the following examples which are set forth to illustrate, but are not to be construed to limit the present invention.

### EXAMPLES

### Example 1

### General Methods

The proteins of the present invention are to be used in drug screening assays, in cell-based or cell-free systems. Cell-based systems can be based in native cells, i.e., cells that normally express the protein, or based in cells that express the protein, fragments or variants expanded in cell culture.

The proteins of the present invention can be used to identify compounds that modulate the activity of the protein in its natural state, as fragments or as recombinant variants. These compounds can be screened for the ability to bind to the protein and further screened against a functional protein to determine the effect of the compound on the protein's activity. These compounds can be tested in animal systems to determine activity/effectiveness. Compounds may be identified that activate (agonist) or inactivate (antagonist) the protein to a desired degree. Agonists can be used to increase the activity of the protein. Antagonists can be used to inhibit the activity of the protein. Candidate compounds may be a variety of chemical entities, such as: soluble peptides, phosphopeptides, antibodies, or small organic and inorganic molecules (e.g., molecules obtained from combinatorial and natural product libraries).

The proteins of the present invention can be used to screen for compounds that stimulate or inhibit interaction between the protein and an entity that normally interacts with the protein. For example, the protein of the present invention is combined with a candidate compound under conditions that allow the protein, fragment, or variant to interact with the target molecule and allows detection of the formation of a complex between the protein and the target, or allows detection of the biochemical consequence of the interaction with the protein and the target.

For example, such an assay would allow for the identification of molecules that modulate the activity of acetyl-CoA carboxylase (ACC). ACC is one of key regulatory steps modulating lipolysis and lipogenesis. ACC carboxylates acetyl-CoA to form malonyl-CoA. In a typical assay, purified ACC, ACC fragment, or ACC variant is combined with acetyl-CoA under conditions that favor the formation of malonyl-CoA. This reaction is combined with a test compound and the rates of malonly-CoA formation are measured and compared to control reactions. Compounds that increase the production of malonyl-CoA over control levels would be classified as agonists. Compounds that inhibit the production of malonyl-CoA over control levels would be classified as antagonists. Further screening of such compounds would be done in cell based systems and animal systems to confirm the activity of the compound. Such assays would measure production of malonyl-CoA in cells or in animal tissues.

In cell free drug screening assays, it is often desirable to immobilize either the protein of the present invention, fragment, or variant. Alternatively, the target molecule may be immobilized. Techniques for immobilizing proteins on matrices are well known to those skilled in the art. For example, the proteins of the present invention can be fused to glutathione-S-transferase to create fusion proteins which can be adsorbed onto glutathione sepharose beads or glutathione derivatized microtitre plates. The beads or plates can be combined with candidate compound reaction conditions and the mixture incubated under conditions conductive to complex formation. Typically, fluorescent or radioactive molecules are added to the plate or beads as a measure of binding or enzymatic reaction. For example, radioactively labeled ATP would be a measure of kinase activity. Such procedures allow for high throughput screening of compound libraries.

For example, such a screening method may identify compounds that modulate the activity of adenosine mono-phosphate kinase (AMPK). Activation AMPK phosphorylates ACC and decreases intracellular malonyl-CoA concentrations thereby increasing ketone body production. ACC, the target of the AMPK, may be fused to glutathione-S-transferase and immobilized on a plate. Test compounds are added to the plate in combination with AMPK and radiolabeled ATP under conditions that do not activate AMPK, i.e. low AMP concentration. Plates are washed and then wells counted for the presence of excess radiolabeled ACC, indicating increased activity of AMPK with the compound.

Agents that modulate one of the proteins of the present invention can be identified using one or more of the above assays, alone or in combination. It is generally preferable to use a cell-based or cell free system first and then confirm activity in an animal or other model system. Such model systems are well known in the art and can readily be employed in this context.

### Example 2

### Inhibition or Reduction in the Activity of Acetyl-CoA Carboxylase (ACC)

Measurement of ACC activity is well known to those skilled in the art, and typically done using a [¹⁴C]bicarbonate fixation assay. For example, Kudo et al teach of an assay for ACC activity (Kudo, et al., J Biol Chem (1995) 270:17513-20). As an example, approximately 200 mg of frozen tissue will be homogenized, using a Tekmar homogenizer, for 30 s at 4° C. in 0.4 ml of buffer containing 50 mM Tris-HCl (pH 7.5), 0.25 M mannitol, 1 mM EGTA, 1 mM EDTA, 1 mM dithiothreitol, 50 mM sodium fluoride, 5 mM sodium pyrophosphate, 1 mM phenylmethylsulfonyl fluoride, 1 mM benzamidine, and 4 μg/ml soybean trypsin inhibitor. The homogenate will be then centrifuged at 14,000 g for 20 min at 4° C., and the resultant supernatant made up to 2.5% (w/v) polyethylene glycol 8000 (PEG 8000) using a stock 25% (w/v) PEG 8000 solution. The solution will be stirred for 10 min, the precipitate removed by centrifugation (10,000 g for 10 min), and the supernatant made up to 6% PEG 8000. After stirring and centrifugation as before, the pellet will be washed with a 6% PEG 8000/homogenizing buffer and be resuspended in 100 mM Tris-HCl (pH 7.5), 50 mM sodium fluoride, 5 mM sodium pyrophosphate, 1 mM EDTA, 1 mM EGTA, 1 mM dithiothreitol, 0.02% sodium azide, 1 mM benzamidine, 4 μg/ml soybean trypsin inhibitor, and 10% glycerol. Protein content will be measured using the BCA method. Acetyl-CoA carboxylase activity in the 6% PEG 8000 fraction will be determined using the [¹⁴C]bicarbonate fixation assay as reported in Witters, et al., Proc. Natl. Acad. Sci. U.S.A. (1988) 86,5473-5477. The assay mixture will contain 60.6 mM Tris acetate (pH 7.5), 1 mg/ml bovine serum albumin, 1.3 μM 2-mercaptoethanol, 2.1 mM ATP, 1.1 mM acetyl-CoA, 5 mM magnesium acetate, 18.2 mM NaH¹⁴CO₃ (approximately 1000 dpm/nmol), and 25 μg of the 6% PEG 8000 pellet. Following a 2-min incubation at 37° C., in the absence or presence of 10 mM citrate, the reaction will be stopped by adding 25 μl of 10% perchloric acid, then centrifuged at 2000 g for 20 min. Radioactivity of supernatant will be determined using standard liquid scintillation counting procedures.

### Example 3

### CPTI Activity

Assays for CPTI activity are well known to those skilled in the art. For example, Vries et al teach a typical method to determine CPT activity (de Vries, et al., Biochemistry (1997) 36:5285-92). Briefly, CPT activity will be assayed by the forward exchange method using L-[³H]carnitine. In a total volume of 0.5 mL, the standard enzyme assay mixture contained 0.2 mM L-[³H]carnitine (˜10 000 dpm/nmol), 50 μM palmitoyl-CoA, 20 mM HEPES (pH 7.0), 1 or 2% fatty acid-free albumin, and 40-75 mM KCl, with or without 10-100 μM malonyl-CoA. Reactions will be initiated by addition of mitochondria, membranes containing expressed proteins, detergent extracts, or proteoliposomes containing the reconstituted CPTI. The reaction will be linear up to 4 min, and all incubations will be done at 30° C. for 3 min. Reactions will be stopped by addition of 6% perchloric acid and will be then centrifuged at 2000 rpm for 7 min. The resulting pellet will be suspended in water, and the product [³H]palmitoylcarnitine will be extracted with butanol at low pH. After centrifugation at 2000 rpm for 2 min, an aliquot of the butanol phase will be transferred to a vial for radioactive counting.

### Example 4

### Malonyl-CoA Binding to CPT

Malonyl-CoA binding assays are well known to those skilled in the art. For example, Shi et al teach a method for measuring binding of malonyl-CoA to CPT (Shi, et al., J Biol Chem (1999) 274:9421-6). In this method, [¹⁴C]Malonyl-CoA binding will be determined by a centrifugation assay. Isolated mitochondria will be suspended in 0.5 ml of ice-cold medium composed of 72 mM sorbitol, 60 mM KCl, 25 mM Tris/HCl (pH 6.8), 1.0 mM EDTA, 1.0 mM dithiothreitol, and 1.3 mg/ml fatty acid-free bovine serum albumin. This will be followed by addition of 0.1-1000 μM [2-¹⁴C]malonyl-CoA, and the suspension will be incubated at 4° C. for 30 min with periodic vortexing. The CPT activity and C₅₀ values are given as the means±S.D. for at least three independent assays with different preparations of mitochondria. The K_{D} values are averages of at least two independent experiments.

### Example 5

### Increase Intracellular Concentration of Carnitine

Assays for the measurement of intracellular carnitine and carnitine transport are well known to those skilled in the art. For example, Wang et al teach a method to measure carnitine transport (Wang, et al., J Biol Chem (2000) 275:20782-6). In this method, Chinese hamster ovary (CHO) cells will be grown in Ham's F-12 medium supplemented with 6% fetal bovine serum. Carnitine transport will be measured at 37° C. with the cluster-tray method. Cells will be grown to confluence in 24-well plates (Costar Corp.) and depleted of intracellular amino acids by incubation for 90 min in Earle's balanced salt solution containing 5.5 mM D-glucose and supplemented with 0.1% bovine serum albumin. Carnitine (0.5 μM, 0.5 μCi/ml) will be then added to the cells for 10 min. Nonsaturable carnitine transport will be measured in the presence of 2 mM unlabeled carnitine. The transport reaction will be stopped by rapidly washing the cells four times with ice-cold 0.1 M MgCl₂. Intracellular carnitine will be then corrected for intracellular water content and expressed as nmol/ml of cell water. Saturable carnitine transport will be calculated by subtracting sodium-independent carnitine transport from total transport, and values are reported as means±S.E. of three to six independent determinations. Carnitine transport in the absence of sodium will be measured substituting methylglucamine for sodium so that the sum of methylglucamine and sodium remained constant at 150 mM. It appears that carnitine accumulation at 0.5 μM will be linear for up to 30 min in cells expressing the normal OCTN2 transporter and for up to 4 h in cells expressing mutant transporters, with a roughly inverse correlation between transport activity and time during which transport remained linear.

Kinetic constants for carnitine transport will be determined by nonlinear regression analysis according to a Michaelis-Menten equation. Na⁺-independent carnitine transport will be determined in parallel trays and subtracted from total transport to obtain Na⁺-dependent carnitine transport. Nonlinear parameters are expressed as means±95% confidence intervals. The Kₘ for sodium (K_{Na}) will be calculated from the intersection (−1/K_{Na}) of linear regressions of 1/v versus 1/[sodium] at three different carnitine concentrations.

### Example 6

### Decrease Activity of Insulin

Insulin signals through a receptor tyrosine kinase. Assays for such activity are well known to those skilled in the art. For example, Qureshi et al teach a method to measure tyrosine kinase activity of the insulin receptor (IRTK) (Qureshi, et al., J Biol Chem (2000) 275:36590-5). To determine IRTK in a cell-free assay, insulin receptor will be partially purified from CHO.IR cells using WGA-agarose columns. For the in vitro kinase assay, 2 μg of WGA-purified insulin receptor will be incubated in a buffer (final volume 50 μl) containing 5 mM MnCl₂, 50 mM HEPES (pH 7.5), 0.1% Triton X-100, insulin or test compounds at 25° C. for 20 min. ATP (25 μM, 0.25 μCi/μl) will be added and incubation continued for 20 min. The mixture will be then incubated for 5 min at 25° C. with 100 μM concentration of a peptide substrate based on insulin receptor autophosphorylation sites (TRDIYETDYYRK). The reaction will be terminated by addition of 10 μl of 1% bovine serum albumin followed by 30 μl of 20% trichloroacetic acid. The mixtures will be centrifuged, and 20 μl of the supernatant will be applied to phosphocellulose filter strip. The filters will be washed several times with 20% trichloroacetic acid, and radioactivity will be determined in a scintillation counter. To determine the activity of recombinant IRTK, a GST fusion protein containing the 48-kDa intracellular domain of insulin receptor (5 nM) will be incubated in a buffer containing 50 mM HEPES (pH 7.5), 10 mM MgCl₂, and 0.1% Triton X-100, test compounds, and ATP (0-200 μM) at 25° C. for 30 min. Biotinylated insulin receptor peptide substrate (above) will be added and the reaction continued for 30 min. The reaction will be then terminated, and IRTK activity will be determined by measuring tyrosine phosphorylation of the insulin receptor peptide substrate using anti-phosphotyrosine antibody in a coupled fluorescence resonance energy transfer reaction according to standard methodology (Zhou, et al., Mol. Endocrinol. (1998) 12, 1594-1604).

### Example 7

### Increase Activity or Levels of Glucagon

Glucagon assays are well known to those skilled in the art. For example, Ling et al. teach a method for measuring glucagons binding to it's receptor and cAMP accumulation (Ling, et al., J Med Chem (2001) 44:3141-9). These binding assays will be carried out in duplicate in polypropylene tubes. The buffer will consist of 25 mM HEPES (pH 7.4) and 0.1% BSA. A total of 100 μL of test compound and 100 μL of [¹²⁵I]glucagon (˜25 000 cpm) will be added to the tubes. Next, 100 μL (˜0.5 μg) of plasma membrane from BHK cells transfected with the cloned human glucagon receptor will be added to the tubes to initiate the assay, and the binding proceeded for 1 h at 37° C. Bound and unbound radioligand will be then separated by vacuum filtration on a Brandel harvester, and the GF/C filters will be counted in a scintillation counter.

### Example 8

### cAMP Accumulation Assay

The cAMP assay will be carried out in borosilicate glass tubes. The buffer will consist of 10 mM HEPES (pH 7.4), 1 mM EGTA, 1.4 mM MgCl₂, 0.1 mM IBMX, 30 mM NaCl, 4.7 mM KCl, 2.5 mM NaH₂PO₄, 3 mM glucose, and 0.2% BSA. BHK cells transfected with the cloned human glucagon receptor (0.5 mL, 10⁶/mL) will be pretreated with various concentrations of compounds for 10 min at 37° C., then challenged with increasing concentrations of glucagon for 20 min. Alternatively, the cells will be treated with various concentrations of the compounds alone to determine if any of the compounds behaved as agonists or antagonists. The reactions will be terminated by centrifugation, followed by cell lysis by the addition of 500 μL of 0.1% HCl. Cellular debris will be pelleted and the supernatant evaporated to dryness. cAMP will be measured by using an RIA kit (NEN).

### Example 9

### Increase the Activity of Apolipoprotein C2 and Lipoprotein Lipase

Assays for lipoprotein lipase (LPL) activity are well known to those skilled in the art. For example, Cruz et al describe a method for determining LPL activity (Cruz, et al., J Biol Chem (2001) 276:12162-8). In this assay, lipase activity will be measured by an in vitro assay in which radiolabeled fatty acids esterified to glycerol are cleaved and recovered after a chloroform/methanol/heptane-based extraction. The units of activity are reported as moles of free fatty acid released per specific number of islets or cells per unit time. LpL activity is distinguishable from other lipase activities by its sensitivity to high molar salt concentration. “Heparin-releasable” LpL activity is the amount of activity in the supernatant of heparin-treated islets or cells. “Detergent-extractable” is the amount of activity after detergent solubilization of remaining cells or islet pellets following heparin treatment. Detergent solubilization involves incubating β-cells or islet pellets with a detergent solution containing 2.0 g/liter deoxycholate for 30 min at 37° C. “Total LpL activity” is the amount of activity after detergent solubilization of cells or islet pellets that have not been exposed to heparin-treatment.

### Example 10

### Activation of Cannabinoid Receptors

Activation of cannabinoid receptors causes a transient Ca²⁺ release, and this has been used to develop assasys for activation of cannabinoid receptors. For example, Sugiura et al. teach a method to assasy cannabinoid receptor activity (Sugiura, et al., J Biol Chem (2000) 275:605-12). To perform this assay, HL-60 cells will be grown at 37° C. in RPMI 1640 medium supplemented with 10% fetal bovine serum in an atmosphere of 95% air and 5% CO₂. Subconfluent cells will be further incubated in fresh medium without fetal bovine serum for 24 h. The cells will be next suspended by gentle pipetting in 25 mM Hepes-buffered Tyrode's solution (−Ca ²⁺) (pH 7.4) containing 3 μM Fura-2/AM and further incubated at 37° C. for 45 min. The cells will be then centrifuged (180×g for 5 min), washed twice with Hepes-Tyrode's solution (−Ca²⁺), and resuspended in Hepes-Tyrode's solution (−Ca²⁺) containing 0.1% BSA. [Ca²⁺], will be estimated using a CAF-100 Ca²⁺ analyzer (JASCO, Tokyo, Japan) as described previously (Sugiura, et al. (1996) Biochem. Biophys. Res. Commun. 229:58-64; Sugiura, et al., J. Biochem. (Tokyo) (1997) 122:890-895; Sugiura, J. Biol. Chem. (1999) 274, 2794-2801). CaCl₂ will be added 4-5 min before the measurement (final Ca²⁺ concentration in the cuvette, 1 mM). 2-AG and other related compounds will be dissolved in dimethyl sulfoxide (Me₂SO), and aliquots (1 μl each) will be added to the cuvette (final Me₂SO concentration, 0.2%). Me₂SO (final concentration, 0.4%) per se did not markedly affect the [Ca²+]. In some experiments, cells suspended in 500 μl of Hepes-Tyrode's solution (−Ca²⁺) containing 0.1% BSA will be pretreated with CP55940 (final concentration, 10 μM) or 2-arachidonoylglycerol (final concentration, 10 μM) or the vehicle alone (1 μl of Me₂SO) at 37° C. for 1 min. Cells will be then sedimented by centrifugation and resuspended in Hepes-Tyrode's solution (−Ca⁺²) containing 0.1% BSA. After the addition of CaCl₂ (final concentration, 1 mM), 2-AG (final concentration, 1 μM) will be added to the cuvette, and the changes in [Ca²⁺], will be analyzed. To examine the effect of the removal of extracellular free Ca²⁺, cells will be incubated in Hepes-Tyrode's solution containing 1 mM CaCl₂ at 37° C. for 3 min. The cell suspension will be then centrifuged, and the supernatant will be removed. The sedimented cells will be resuspended in Hepes-Tyrode's solution containing 0.1 mM EGTA. The effect of 2-AG (final concentration, 1 μM) on [Ca²⁺], will be analyzed as described above.

### Example 11

### Activate Adenosine Mono-Phosphate Kinase (AMPK)

Measurement of AMPK activity is well known to those skilled in the art, and typically done by measuring incorporation of labeled phosphate into a substrate molecule. For example, Kudo et al teach a method of assaying AMPK activity in tissue. Approximately 200 mg of frozen tissue will be homogenized, using a Tekmar homogenizer, for 30 s at 4° C. in 0.4 ml of buffer containing 50 mM Tris-HCl (pH 7.5), 0.25 M mannitol, 1 mM EGTA, 1 mM EDTA, 1 mM dithiothreitol, 50 mM sodium fluoride, 5 mM sodium pyrophosphate, 1 mM phenylmethylsulfonyl fluoride, 1 mM benzamidine, and 4 μg/ml soybean trypsin inhibitor. The homogenate will be then centrifuged at 14,000 g for 20 min at 4° C., and the resultant supernatant made up to 2.5% (w/v) polyethylene glycol 8000 (PEG 8000) using a stock 25% (w/v) PEG 8000 solution. The solution will be stirred for 10 min, the precipitate removed by centrifugation (10,000 g for 10 min), and the supernatant made up to 6% PEG 8000. After stirring and centrifugation as before, the pellet will be washed with a 6% PEG 8000/homogenizing buffer and resuspended in 100 mM Tris-HCl (pH 7.5), 50 mM sodium fluoride, 5 mM sodium pyrophosphate, 1 mM EDTA, 1 mM EGTA, 1 mM dithiothreitol, 0.02% sodium azide, 1 mM benzamidine, 4 μg/ml soybean trypsin inhibitor, and 10% glycerol. Protein content will be measured using the BCA method. AMPK will be assayed in the 6% PEG 8000 fraction by following the incorporation of ³²P into a synthetic peptide (termed SAMS peptide) with the amino acid sequence HMRSAMSGLHLVKRR. The assay will be performed in a 25 μl total volume containing 40 mM HEPES-NaOH (pH 7.0), 80 mM NaCl, 8% glycerol, 0.8 mM EDTA, 200 μM SAMS peptide, 0.8 mM dithiothreitol, 5 mM MgCl₂, 200 μM [γ-³²P]ATP (400-600 dpm/pmol), and 6-8 μg of the 6% PEG 8000 pellet. The assay will be performed in the absence or presence of 200 μM 5′-AMP at 30° C. for 5 min. The reaction will be initiated by the addition of [³²P]ATP/Mg. At the end of the incubation, 15-μl aliquots will be removed and spotted on 1×1-cm square of phosphocellulose paper (P81, Whatman), which will be subsequently placed into 500 ml of 150 mM H₃PO₄. These papers will be washed 4 times for 30 min with 150 mM H₃PO₄, and then washed 20 min with acetone. The papers will be then dried and placed in vials containing 4 ml of scintillant. Radioactivity will be determined using standard liquid scintillation procedures.

### Example 12

### Increasing Uptake of Ketone Bodies by the Brain

Assays for activity of monocarboxylate transporters are well known to those skilled in the art. For Example, Broer et al. teach a method to measure lactate (a monocarboxylate) uptake in rat astroglial cells (Broer, et al., J Biol Chem (1997) 272:30096-102). For uptake experiments, rat astroglial cells will be grown to a density of 4×10⁶ per 60-mm culture dish in a humidified atmosphere of 10% CO₂ in air at 37° C. in 90% Dulbecco's modified Eagle's medium, 10% fetal calf serum containing 44 mM NaHCO₃. All experiments will be performed at 21° C. Growth medium will be aspirated, and cells will be washed three times with 3 ml of HBSS (136.6 mM NaCl, 5.4 mM KCl, 4.0 mM HEPES, 2.7 mM Na₂HPO₄, 1 mM CaCl₂, 0.5 mM MgCl₂, 0.44 mM KH₂PO₄, 0.41 mM MgSO₄, pH 7.8). To reduce metabolism of lactate, cells will be preincubated for 5 min with 1 mM aminooxyacetate in HBSS. To initiate transport, the preincubation medium will be aspirated and replaced by 3 ml of HBSS containing 1 mM aminooxyacetate, [¹⁴C]lactate, and unlabeled lactate at different concentrations resulting in a specific activity of 500 dpm/nmol. After 15 s, transport will be stopped by aspirating the transport buffer followed by three washing cycles with 3 ml of ice-cold HBSS. Cells will be lysed by addition of 1 ml of 0.1 M HCl. Of the resulting suspension an aliquot portion of 900 μl will be mixed with 3 ml of scintillation mixture, and radioactivity will be determined in a scintillation counter. An aliquot portion of 100 μl will be used for protein determination using the Bio-Rad Protein assay (Bio-Rad Laboratories, München, Germany).

What is claimed is:

1. A method of identifying an agent that increases the output of ketone bodies by hepatocytes comprising contacting a hepatic cell or hepatic-derived cell with at least one candidate agent, and detecting the output of ketone bodies by the cell, whereby the agent is identified.

2. The method of claim 1, wherein the detecting the output of ketone bodies comprises detecting genomic changes, cytochemical changes, mRNA changes, or protein changes, or metabolic changes.

3. The method of claim 1, wherein said candidate agent is suspected of modulating the function of acetyl-CoA carboxylase, and wherein said detecting the output of ketone bodies comprises detecting the activity of hepatic acetyl-CoA carboxylase.

4. The method of claim 3, wherein said candidate agent is suspected of inhibiting the action of insulin on acetyl-CoA carboxylase.

5. The method of claim 3, wherein candidate agent is suspected of modulating the intracellular level of glucagon.

6. The method of claim 1, wherein said candidate agent is suspected of modulating the binding of malonyl-CoA to carnitine palmitoyl-transferase I.

7. The method of claim 1, wherein candidate agent is suspected of modulating the availability of carnitine.

8. A method of identifying an agent that increases the output of ketone bodies by astrocytes comprising contacting an astrocyte or astrocyte-derived cell with at least one candidate agent, and detecting the output of ketone bodies or activity of the target of the cell, whereby the agent is identified.

9. The method of claim 8, wherein said candidate agent is suspected of modulating the function of apoC2, and wherein said detecting the output of ketone bodies comprises detecting the activity of apoC2.

10. The method of claim 9, wherein detecting the activity of apoC2 comprises detecting E4 binding to VLDL.

11. The method of claim 8, wherein said candidate agent is suspected of modulating the function of cannabinoid receptors, and wherein said detecting the output of ketone bodies comprises detecting the activity of cannabinoid receptors.

12. The method of claim 8, wherein said candidate agent is suspected of modulating the function of lipoprotein lipase, and wherein said detecting the output of ketone bodies comprises detecting the activity of lipoprotein lipase.

13. The method of claim 12, wherein detecting the activity of lipoprotein lipase comprises detecting C2 binding to VLDL.

14. The method of claim 9, wherein said candidate agent is suspected of modulating the binding of malonyl-CoA to carnitine palmitoyl-transferase I.

15. The method of claim 14, wherein candidate agent is suspected of modulating the availability of carnitine.

16. The method of claim 8, wherein said candidate agent is suspected of modulating the activity of acetyl-CoA carboxylase.

17. The method of claim 8, wherein said candidate agent is suspected of modulating the activity of adenosine monophosphate kinase.

18. A method of identifying an agent that increases the uptake of ketone bodies by a component selected from the group consisting of an astrocyte, and astrocyte-derived cell, non-neonatal brain, and non-neonatal brain tissue, comprising contacting said component with at least one candidate agent, and detecting the uptake of ketone bodies by said component, whereby the agent is identified.

19. The method of claim 18, wherein said candidate agent is suspected of modulating the levels or activity of the monocarboxylate transporter family of proteins.
